(19) United States
(12) Patent Application Publication
(10) Pub. No.: US 2026/0108549 A1
(43) Pub. Date: Apr. 23, 2026

(54) MACROLIDE COMPOUND AND PREPARATION METHOD AND APPLICATION THEREOF

(71) Applicant: INSTITUTE OF FEED RESEARCH, CHINESE ACADEMY OF AGRICULTURAL SCIENCE, Beijing (CN)

(72) Inventors: Xiubo LI, Beijing (CN); Fei XU, Beijing (CN); Yiming LIU, Beijing (CN); Xiaojie CHEN, Beijing (CN)

(73) Assignee: INSTITUTE OF FEED RESEARCH, CHINESE ACADEMY OF AGRICULTURAL SCIENCE, Beijing (CN)

(21) Appl. No.: 19/469,376

(22) PCT Filed: May 31, 2024
(86) PCT No.: PCT/CN2024/096706

§ 371 Date: Sep. 26, 2025

(30) Foreign Application Priority Data
Nov. 30, 2023 (CN) 202311620585.2

(51) Int. Cl. A61K 31/7048; A61P 31/04; C07H 17/08

(52) U.S. Cl. CPC A61K 31/7048; A61P 31/04; C07H 17/08

(57) ABSTRACT

A macrolide compound and a preparation method and application thereof are provided. The macrolide compound has a structure represented by Formula I. A pharmaceutical composition or a veterinary pharmaceutical composition, which includes the macrolide compound with a structure represented by Formula I, is also provided. Moreover, a pharmaceutical preparation, which includes the macrolide compound with a structure represented by Formula I, is provided. The macrolide compound, veterinary pharmaceutical composition and pharmaceutical preparation can be used to treat pathogen infections, especially to treat pathogen infections in animals, with a significant effect.

### CROSS REFERENCE TO THE RELATED APPLICATIONS

This application is the national phase entry of International Application No. PCT/CN2024/096706, filed on May 31, 2024, which is based upon and claims priority to Chinese Patent Application No. 202311620585.2, filed on Nov. 30, 2023, the entire contents of which are incorporated herein by reference.

### TECHNICAL FIELD

The present invention relates to the technical field of the synthesis of heterocyclic compounds and animal pharmacy, and in particular to a macrolide compound and a preparation method and application thereof.

### BACKGROUND

Macrolide compounds are an important class of antibiotics with advantages such as good anti-infective properties, few side effects, etc. They are widely used in livestock and poultry veterinary clinics (Wang Xiuru, Properties, characteristics and applications of macrolide antibiotics. Veterinary Orientation 2018, 54-55. Kuang Baoxiao. A new antibiotic for animals-Tildipirosin. Pigs Today, 2022, 98-100.). Macrolide antibiotics are classified according to their chemical structures as 14-membered, 15-membered and 16-membered macrolide antibiotics. The main varieties include Erythromycin, Azithromycin, Kitasamycin, Tylosin, Tilmicosin, Tylvalosin, Gamithromycin, etc. (Yu Zhichao. Research progress of veterinary antibiotic Gamithromycin. Modern Animal Husbandry 2020, 22-23. Liu Wang; Pei Wei. The latest research progress of Azithromycin. Chinese Journal of Animal Husbandry and Veterinary Medicine 2014, 15.). They are mainly used to treat bovine respiratory diseases, mastitis, arthritis and otitis media caused by bovine mycoplasma infection, swine asthma caused by Mycoplasma hyopneumoniae, porcine proliferative enteritis caused by Lawsonia intracellularis, and chicken chronic respiratory diseases caused by Mycoplasma gallisepticum (He Chengguang; Kong Lingcong; Sun Zhe. Research progress on antibiotic resistance of bovine mycoplasma. Jilin Animal Husbandry and Veterinary Medicine 2018, 39, 11-13. Wan Jin; Ma Nini; Wang Cong. Research progress on the efficacy of tylvalosin tartrate in the prevention and treatment of specific livestock and poultry diseases. China Animal Health Inspection, 2021, 38, 75-78.).

Tylosin is an important macrolide antibiotic for livestock and poultry, which has a 16-membered macrolide structure and was first extracted from the culture medium of Streptomyces fradiae in 1959. The veterinary clinical products used for livestock and poultry mainly include tylosin tartrate, tylosin lactate, tylosin sulfate, tylosin hydrochloride and tylosin phosphate (Chen Dong; Zhang Xiaoqiang; Na Qi; Suo Jiawei. Study on optimization of tylosin purification process. Modern Chemical Research, 2023, 170-172. Liu Jia; Hao Shengyan; Pan Faming. Progress in research on determination of Tylosin residues in animal products. Animal Husbandry & Veterinary Medicine 2022, 54, 148-152.). Tylosin has good antibacterial activity against pathogens such as Gram-negative bacteria, Gram-positive bacteria, mycoplasma, etc.; it can not only be used to treat swine dysentery, poultry mycoplasma infection, pneumonia in ruminant and other diseases, but also can be used as a feed additive to promote animal growth (Wang Lixia; Li Shenglong; Chen Dangtong; Wang Jun. Establishment of high performance liquid chromatography detection method for tylosin. Journal of Anhui Agricultural Sciences 2020, 48, 206-209.).

In order to develop new macrolide antibiotics, domestic and foreign scholars have made various modifications to the structure of tylosin and have synthesized a series of tylosin derivatives (Zhao Dongfeng; Ren Xiang; Zhu Li. Research progress of tylosin and its derivatives. China Medical Herald, 2006, 46-48.). For example, 10,11,12,13-tetrahydrodesmycosin derivatives (Narandja, A.; Kelneric, K.; Kolacny-Babic, L.; Djokic, S. 10,11,12,13-Tetrahydro Derivatives of Tylosin. Ii. Synthesis, Antibacterial Activity and Tissue Distribution of 4′-Deoxy-10,11,12,13-Tetrahydrodesmycosin. Journal of Antibiotics. 1995, 48, 248-253. Narandja, A.; Djokic, S. Derivatives of 10,11,12,13-tetra-hydrodesmycosin, processes for preparation, and use thereof in obtaining pharmaceuticals. Patent EP0490311, 1992-06-17), 9-oxime tylosin derivatives (Wang Huanhuan; Yang Pu; Zhai Hongjin; Zhang Shuo; Cao Yaquan; Yang Yingxue; Wu Chunli Design, synthesis and activity evaluation of new tylosin derivatives. Chinese Journal of Organic Chemistry 2022, 42, 557-571.), 12,13-epoxy-tylosin (Narandja, A.; Lopotar, N. Derivatives of 12,13-Epoxy-tylosin and processes of manufacture thereof. Patent U.S. Pat. No. 5,688,924, 1997-11-18), Tildipirosin (Zhang, C.; Song, M.; Qi, P.; Zhang, G.; Ge, X.; Zhao, M.; Wu, J.; Ma, J.; Wang, D.; Process for preparation of 20,23-dipiperidinyl-5-O-mycaminosyl-tylonolide. Patent CN 104892704 B, 2017-08-08.), Tylvalosin (Research progress on the efficacy of Tylvalosin tartrate in the prevention and treatment of specific livestock and poultry diseases. China Animal Health Inspection, 2021, 38, 75-78.), etc.

### SUMMARY

The object of the present invention is to provide new macrolide compounds and a preparation method and application thereof. The compound can be used to treat or prevent pathogen infections in animals with a significant effect.

To achieve the above object, the present invention provides the following technical solutions:

In a first aspect, the present invention provides a macrolide compound having a structure represented by Formula I:

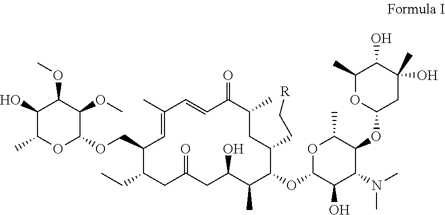

wherein R is selected from the group consisting of 2-hydroxyethylamino, 3-hydroxypropylamino, di(2-hydroxyethylamino), di(3-hydroxypropylamino), (R)-2-hydroxymethyltetrahydropyrrolyl, (S)-2-hydroxymethyltetrahydropyrrolyl, 4-hydroxypiperidyl, and 4-hydroxymethylpiperidyl.

Specifically, the macrolide compound is: 20-(2-hydroxyethylamino) tylosin, 20-(3-hydroxypropylamino) tylosin, 20-(bis(2-hydroxyethylamino)) tylosin, 20-(bis(3-hydroxypropylamino)) tylosin, 20-((R)-2-hydroxymethyltetrahydropyrrolyl) tylosin, 20-((S)-2-hydroxymethyltetrahydropyrrolyl) tylosin, 20-(4-hydroxypiperidyl) tylosin, 20-(4-hydroxymethylpiperidyl) tylosin.

The macrolide compound has a preferred structure as shown in any one of Formula Ia, Formula Ib, Formula Ic or Formula Id:

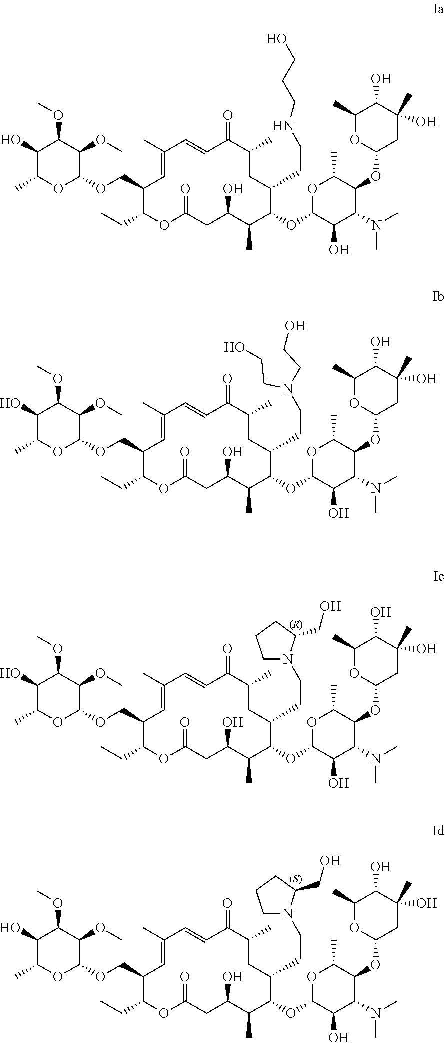

The present invention also provides pharmaceutically acceptable salts of the macrolide compounds as described above.

The pharmaceutically acceptable salt refers to a salt formed by the macrolide compound with an acid.

The acid includes: tartaric acid, hydrochloric acid, phosphoric acid, sulfuric acid, salicylic acid, methanesulfonic acid, lactic acid, malic acid, formic acid, acetic acid, propionic acid, fumaric acid, citric acid, oxalate, maleic acid, succinic acid, benzoic acid, ethanedisulfonic acid and the like.

In a second aspect, the present invention further provides a preparation method of the macrolide compound as described above, including the following steps of:
S1. reacting tylosin A with an amino alcohol;
S2. adding a reducing agent or an acid to the system obtained by the reaction in step S1, and reacting to obtain the macrolide compound.

Further, the preparation method includes a synthesis route 1 and a synthesis route 2: The synthesis route 1 includes the following steps of:
(1) reacting the tylosin A with the amino alcohol in a polar solvent to obtain an imine solution;
(2) adding a reducing agent to the imine solution, and reacting to obtain a hydroxyl secondary amino-modified macrolide compound.

In step (1), the amino alcohol is 2-aminoethanol, 3-aminopropanol.

In step (1), the molar ratio of the amino alcohol to the tylosin A is from 2:1 to 5:1, preferably 3:1 to 3.5:1.

In step (1), the polar solvent is selected from one or more of the group consisting of methanol, ethanol, propanol, isopropanol, n-butanol and ethylene glycol.

In step (1), the reaction conditions include: a temperature of room temperature, and a time period of 12-13 h.

In step (2), the reducing agent is selected from one or more of the group consisting of sodium borohydride, sodium triacetoxyborohydride and LiAlH₄.

In step (2), the molar ratio of the reducing agent to the tylosin A is from 1:4 to 1:1, preferably 2:1 to 2.5:1.

In step (2), the reaction conditions include: a temperature of room temperature and a time period of 2 to 3 h.

Further, the synthesis route 1 further includes: before adding the reducing agent, TLC is performed to monitor the reaction to ensure that the raw material is completely converted into imine.

Further, the synthesis route 1 also includes post-treatment steps. The post-treatment is carried out in accordance with the following operations: adding an aqueous solution of a base into the reaction system to quench the reaction, and then concentrating it under reduced pressure to remove the alcohol solvent; extracting the remaining aqueous solution with an organic solvent, washing the combined organic phase with a saturated brine, drying it over anhydrous sodium sulfate, and concentrating it under reduced pressure; wherein the base is selected from one or more of the group consisting of potassium carbonate, sodium carbonate, potassium hydroxide or sodium hydroxide; and the organic solvent is selected from one or more of the group consisting of dichloromethane, ethyl acetate or diethyl ether.

By way of example, the synthesis route of the synthesis route 1 as described above is as follows:

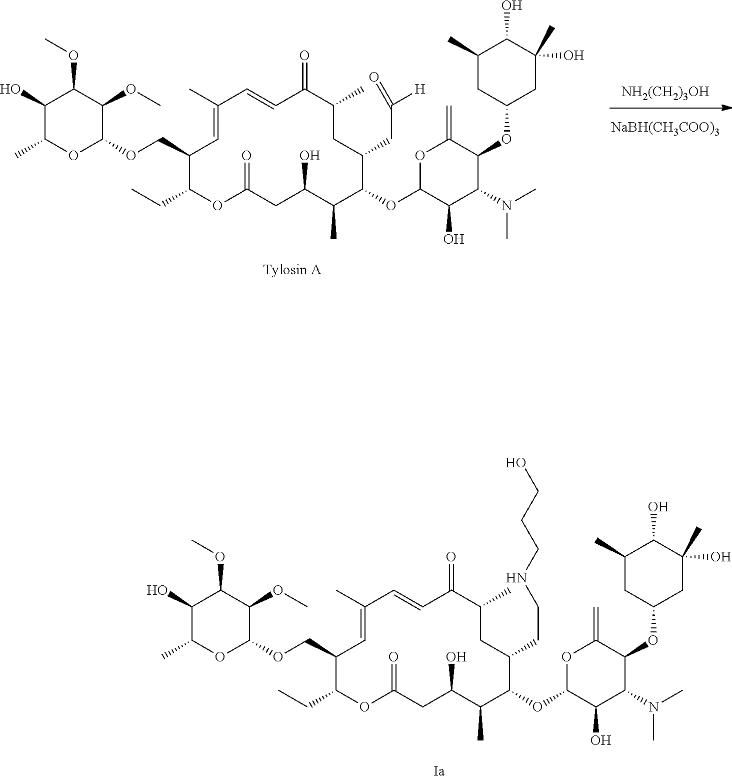

The synthesis route 2 includes the following steps of:
(A) reacting the tylosin A with the amino alcohol in a non-polar solvent to obtain a reaction mixture;
(B) adding an acid to the reaction mixture obtained in step (A), and reacting to obtain a hydroxyl tertiary amino-modified macrolide compound.

In step (A), the amino alcohol is 2-aminoethanol, 3-aminopropanol, (R)-prolinol, (S)-prolinol, 4-hydroxypiperidine, 4-hydroxymethylpiperidine.

In step (A), the molar ratio of the amino alcohol to the tylosin A is from 2:1 to 5:1, preferably 2.5:1 to 3.5:1.

In step (A), the non-polar solvent is selected from one or more of the group consisting of dimethoxyethane, benzene and toluene.

In step (B), the acid is formic acid.

In step (B), the acid is added when the temperature of the reaction system reaches 75-85° C., preferably 80° C.

In step (B), the molar ratio of the acid to the tylosin A is from 3:1 to 6:1, preferably 5:1 to 6:1.

In step (B), the reaction conditions include: a temperature of 78-80° C. and a time period of 2-2.5 h.

Further, the synthesis route 2 also includes post-treatment steps; and the post-treatment is carried out in accordance with the following operations: adding distilled water to the reaction system, adjusting the pH of the aqueous phase obtained after the separation of liquid to 9-11 with a base, extracting the aqueous solution with an organic solvent, washing the combined organic phase with a saturated brine, drying it over anhydrous sodium sulfate, and concentrating it under reduced pressure; wherein the base is selected from one or more of the group consisting of potassium carbonate, sodium carbonate, potassium hydroxide or sodium hydroxide; and the organic solvent is selected from one or more of the group consisting of dichloromethane, ethyl acetate or diethyl ether.

Further, the preparation method of the macrolide compound provided by the present invention also includes a purification step: adding the obtained crude product into a silica gel chromatography column, selecting two organic solvents to prepare them into eluents of different polarities, and using gradient elution to remove impurities from the crude product, thereby obtaining a pure product of macrolide compound; wherein the eluents may be selected from any two of the group consisting of diethyl ether, ethyl acetate, methanol, isopropanol, acetone or dichloromethane.

By way of example, the synthesis route of the synthesis route 2 as described above is as follows:

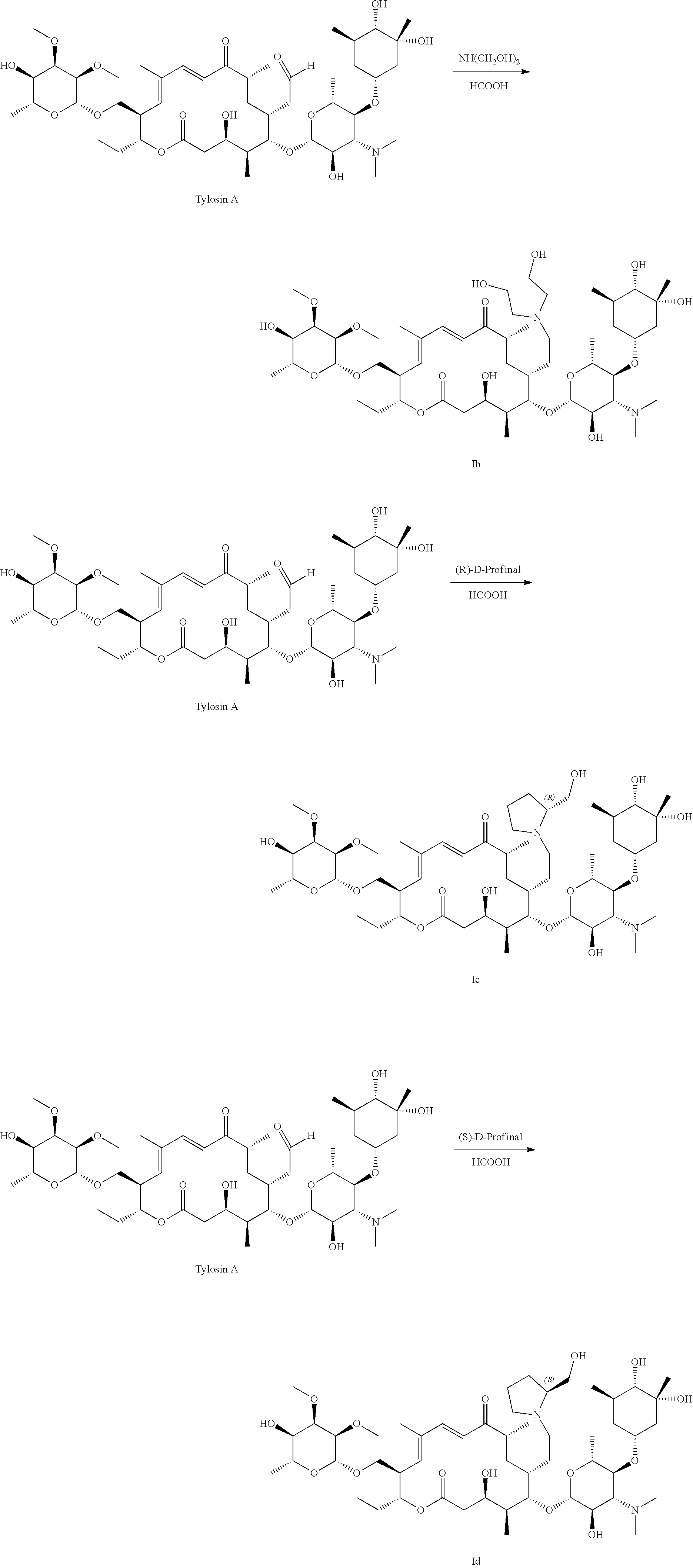

In a third aspect, the present invention further provides a veterinary pharmaceutical composition including the macrolide compound with a structure represented by Formula I as described above.

In a fourth aspect, the present invention further provides a pharmaceutical preparation including the macrolide compound with a structure represented by Formula I as described above.

The pharmaceutical preparation has a dosage form of powder, tablet, premix, soluble powder, injection.

In a fifth aspect, the present invention further provides use of the macrolide compound, the veterinary pharmaceutical composition and the pharmaceutical preparation as described above for preparing anti-pathogen infection pharmaceuticals. By way of example, the anti-pathogen infection pharmaceuticals are products for clinical use in livestock and poultry veterinarians.

In the application, the pathogen is Mycoplasma, Pasteurella, Pasteurella multocida, Staphylococcus aureus, Streptococcus agalactiae, Streptococcus pneumoniae, β-hemolytic Streptococcus, Escherichia coli, Haemophilus influenzae, Actinobacillus pleuropneumoniae, Salmonella, Mannheimia, Erysipelothrix rhusiopathiae.

### DETAILED DESCRIPTION OF THE EMBODIMENTS

The present invention will be further set forth below combined with specific examples. However, the present invention is not limited to the following examples.

Unless otherwise specified, the experimental methods used in the following examples are conventional methods.

Unless otherwise specified, the reagents, materials, instruments and so on used in the following examples are commercially available.

The sources of the raw materials used in the following examples are as follows:

### Example 1

Tylosin A (3.00 g, 3.27 mmol) was added to a 50 mL Schlenk flask, methanol (18 mL) was added, and then 3-amino-1-propanol (0.74 g, 9.85 mmol) was slowly added with a syringe, and the reaction was stirred at room temperature for 12 h.

After TLC detected that the raw material was completely converted into imine, sodium triacetoxyborohydride (1.39 g, 6.56 mmol) was slowly added at room temperature, and stirring of the reaction was continued at room temperature for 2 h.

After TLC detected the completion of the reaction, 1 M aqueous NaOH solution (3 mL) was added to quench the reaction. Then, MeOH was removed by concentration under reduced pressure, and the residue was extracted with dichloromethane (10 mL×3). The combined extract was washed with a saturated aqueous NaCl solution (10 mL) and dried over anhydrous sodium sulfate. Filtration was performed, the filtrate was concentrated under reduced pressure to obtain a crude product. Finally, purification was performed by silica gel column chromatography (dichloromethane/methanol=8:1) to obtain a white solid, the macrolide compound Ia (1.40 g, a yield of 44%).

¹H NMR (500 MHz, CDCl₃) δ 7.37 (d, J=14.7 Hz, 1H), 6.30 (d, J=14.8 Hz, 1H), 5.95 (s, 1H), 5.08 (d, J=9.6 Hz, 1H), 4.96 (d, J=10.6 Hz, 1H), 4.58 (t, J=10.5 Hz, 1H), 4.33-4.25 (s, 4H), 4.09-4.08 (m, 1H), 4.01-3.99 (m, 1H), 3.80-3.75 (m, 4H), 3.67-3.61 (m, 4H), 3.56 (s, 2H), 3.49-3.47 (m, 2H), 3.29 (t, J=10.5 Hz, 2H), 3.19 (d, J=10.4 Hz, 1H), 3.02-2.95 (m, 4H), 2.87-2.80 (m, 3H), 2.72-2.68 (m, 4H), 2.51-2.44 (m, 8H), 2.06-1.95 (m, 3H), 1.87-1.74 (m, 8H), 1.63-1.62 (m, 3H), 1.53-1.48 (m, 2H), 1.31-1.23 (m, 14H), 1.16-1.11 (m, 4H), 1.03 (s, 3H), 0.93 (d, J=8.2 Hz, 3H).

¹³C NMR (126 MHz, CDCl₃) δ 203.84, 173.60, 148.26, 142.99, 134.55, 117.88, 103.71, 101.01, 96.38, 81.69, 79.83, 79.50, 76.32, 75.03, 74.88, 72.84, 72.67, 71.67, 70.34, 69.39, 69.02, 68.70, 66.59, 65.88, 61.88, 61.66, 59.51, 47.26, 46.07, 45.94, 44.97, 41.91, 41.05, 40.85, 39.32, 33.47, 32.41, 30.42, 29.55, 26.14, 25.34, 25.18, 19.03, 18.18, 17.69, 17.52, 12.82, 10.52, 9.55.

TLC R_{f}=0.4 (dichloromethane/methanol=8:1)

HRMS (ESI, m/z): [M+H]⁺ calcd for C₄₉H₈₇N₂O₁₇, 975.59993; found 975.60059.

### Example 2

Tylosin A (0.50 g, 0.55 mmol) was added to a 50 mL three-necked flask equipped with a condenser, toluene (6 mL) was added, and it was stirred for dissolving. Then diethanolamine (0.17 g, 1.62 mmol) was added, the temperature was raised to 80° C., formic acid (0.14 g, 3.04 mmol) was added, and stirring of the reaction was continued at 80° C. for 2 h.

After TLC detected that the reaction was complete, distilled water (5 mL) was added to quench the reaction and the separation of liquid was performed. The aqueous phase was adjusted to pH 10 with 5 M aqueous sodium hydroxide solution and then extracted with dichloromethane (15 mL×3). The combined extract was dried over anhydrous sodium sulfate. Filtration was performed, and the filtrate was concentrated under reduced pressure to obtain a crude product. Finally, purification was performed by silica gel column chromatography (dichloromethane/methanol=8:1) to obtain a white solid, the macrolide compound Ib (0.27 g, a yield of 49%).

¹H NMR (500 MHz, CDCl₃) δ 7.44 (d, J=14.4 Hz, 1H), 6.31 (d, J=15.7 Hz, 1H), 5.98 (s, 1H), 5.08 (d, J=10.2 Hz, 1H), 4.93 (d, J=9.9 Hz, 1H), 4.58 (t, J=9.1 Hz, 1H), 4.33-4.29 (m, 2H), 4.11-4.08 (m, 1H), 4.03-3.99 (m, 1H), 3.79-3.57 (m, 13H), 3.47-3.45 (m, 2H), 3.32-3.28 (m, 2H), 3.21-3.17 (m, 1H), 3.05-2.95 (m, 3H), 2.72-2.60 (m, 7H), 2.51-2.49 (m, 8H), 2.42-2.38 (m, 2H), 2.28-2.26 (m, 1H), 2.06-2.01 (m, 1H), 1.95-1.75 (m, 7H), 1.66-1.59 (m, 3H), 1.50-1.46 (m, 2H), 1.32-1.21 (m, 16H), 1.12-1.05 (m, 7H), 0.96-0.91 (m, 3H).

¹³C NMR (126 MHz, CDCl₃) δ 205.13, 173.62, 149.18, 143.77, 134.55, 117.45, 103.57, 101.03, 96.33, 81.66, 80.09, 79.83, 76.33, 75.15, 75.07, 72.76, 72.68, 71.74, 70.36, 69.40, 69.10, 68.75, 66.33, 65.89, 61.64, 59.58, 59.47, 59.43, 57.17, 53.02, 45.97, 45.94, 45.27, 44.89, 41.93, 41.01, 40.88, 39.39, 33.80, 33.65, 26.06, 25.34, 25.25, 19.10, 18.18, 17.69, 17.46, 12.76, 10.89, 9.66.

TLC R_{f}=0.2 ((dichloromethane/methanol=8:1)

HRMS (ESI, m/z): [M+H]⁺ calcd for C₅₀H₈₉N₂O₁₈, 1005.61049; found 1005.62402.

### Example 3

Tylosin A (0.50 g, 0.55 mmol) was added to a 50 mL three-necked flask equipped with a condenser, toluene (6 mL) was added, then (R)-prolinol (0.17 g, 1.68 mmol) was added, and it was stirred for dissolving. The temperature was raised to 80° C., formic acid (0.14 g, 3.04 mmol) was added, and stirring of the reaction was continued at 80° C. for 2 h.

After TLC detected that the reaction was complete, distilled water (5 mL) was added to quench the reaction and the separation of liquid was performed. The aqueous phase was adjusted to pH 10 with 5 M aqueous sodium hydroxide solution and then extracted with dichloromethane (15 mL×3). The combined extract was dried over anhydrous sodium sulfate. Filtration was performed, and the filtrate was concentrated under reduced pressure to obtain a crude product. Finally, purification was performed by silica gel column chromatography (dichloromethane/methanol=8:1) to obtain a white solid, the macrolide compound Ic (0.32 g, a yield of 58%).

¹H NMR (500 MHz, CDCl₃) δ 7.36 (d, J=17.5 Hz, 1H), 6.30 (d, J=14.8 Hz, 1H), 5.94 (s, 1H), 5.09-5.07 (m, 1H), 4.97-4.94 (m, 1H), 4.58-4.55 (m, 1H), 4.29-4.26 (m, 2H), 4.11-4.06 (m, 1H), 4.02-3.98 (m, 1H), 3.83-3.81 (m, 1H), 3.76-3.73 (m, 1H), 3.62-3.53 (m, 7H), 3.47-3.44 (m, 4H), 3.33-3.29 (m, 2H), 3.19-3.17 (m, 2H), 3.03-2.93 (m, 3H), 2.72-2.63 (m, 3H), 2.59-2.54 (m, 6H), 2.50-2.46 (m, 5H), 2.36-2.26 (m, 2H), 2.04-2.01 (m, 2H), 1.89-1.83 (m, 3H), 1.79-1.72 (m, 7H), 1.63-1.55 (m, 4H), 1.31-1.19 (m, 16H), 1.08-1.06 (m, 5H), 1.01-0.99 (m, 2H), 0.93-0.91 (m, 3H).

¹³C NMR (126 MHz, CDCl₃) δ 203.98, 173.63, 162.63, 161.84, 148.07, 143.06, 134.38, 117.97, 103.97, 101.01, 96.36, 82.44, 81.69, 79.86, 76.32, 75.01, 74.91, 72.95, 72.66, 71.65, 70.35, 69.37, 69.07, 68.80, 66.48, 65.89, 65.12, 63.03, 61.64, 59.50, 55.00, 54.69, 46.01, 45.11, 41.93, 41.63, 40.87, 39.36, 34.95, 34.22, 27.64, 26.75, 25.34, 25.17, 23.48, 19.13, 18.18, 17.69, 12.76, 11.16, 9.60, 9.33.

TLC R_{f}=0.3 (dichloromethane/methanol=8:1)

HRMS (ESI, m/z): [M+H]⁺ calcd for C₅₁H₈₉N₂O₁₇, 1001.61558; found 1001.61896.

### Example 4

Tylosin A (1.00 g, 1.09 mmol) was added into a 50 mL three-necked flask equipped with a condenser, toluene (8 mL) was added, then (S)-prolinol (0.33 g, 3.26 mmol) was added, and it was stirred for dissolving. The temperature was raised to 80° C., formic acid (0.27 g, 5.86 mmol) was added, and the reaction was continued at 80° C. for 2 h.

After TLC detected that the reaction was complete, distilled water (8 mL) was added to quench the reaction and the separation of liquid was performed. The aqueous phase was adjusted to pH 10 with 5 M aqueous sodium hydroxide solution and then extracted with dichloromethane (15 mL×3). The combined extract was dried over anhydrous sodium sulfate. Filtration was performed, and the filtrate was concentrated under reduced pressure to obtain a crude product. Finally, purification was performed by silica gel column chromatography (dichloromethane/methanol=8:1) to obtain a white solid, the macrolide compound Id (0.64 g, a yield of 59%).

¹H NMR (500 MHz, CDCl₃) δ 7.31 (d, J=15.4 Hz, 1H), 6.29 (d, J=15.5 Hz, 1H), 5.92 (s, 1H), 5.11-5.06 (m, 1H), 4.97-4.91 (m, 1H), 4.59-4.55 (m, 1H), 4.32-4.26 (m, 2H), 4.11-4.07 (m, 1H), 4.02-3.97 (m, 1H), 3.76-3.72 (m, 2H), 3.65-3.61 (m, 4H), 3.57-3.53 (m, 3H), 3.50-3.42 (m, 4H), 3.33-3.27 (m, 2H), 3.21-3.17 (m, 2H), 3.02-2.93 (m, 3H), 2.87-2.68 (m, 3H), 2.61-2.42 (m, 13H), 2.16-2.09 (m, 2H), 1.88-1.74 (m, 10H), 1.65-1.56 (m, 4H), 1.33-1.19 (m, 16H), 1.10-1.02 (m, 7H), 0.96-0.90 (m, 3H).

¹³C NMR (126 MHz, CDCl₃) δ 203.54, 172.99, 162.58, 161.82, 147.71, 142.84, 134.41, 117.96, 103.63, 100.97, 96.28, 81.63, 79.82, 79.45, 76.29, 75.01, 74.77, 72.76, 72.64, 71.71, 70.29, 69.33, 69.12, 68.71, 66.41, 65.83, 65.56, 61.65, 61.58, 59.45, 53.48, 53.38, 45.95, 45.14, 41.91, 41.17, 40.84, 39.62, 33.60, 32.58, 26.98, 26.66, 25.31, 23.27, 23.21, 19.05, 18.14, 17.65, 12.79, 11.10, 9.59.

TLC R_{f}=0.3 (dichloromethane/methanol=8:1)

HRMS (ESI, m/z): [M+H]⁺ calcd for C₅₁H₈₉N₂O₁₇, 1001.61558; found 1001.61746.

Test Example 1 Determination of the antibacterial activity of the compounds of the present invention

The antibacterial activity of the compounds obtained in Examples 1-4 of the present invention were determined by using the broth microdilution method with tylosin as a positive control.

The test method is as follows:

A broth culture medium was added into a 96-well plate, a prepared compound solution was diluted two-fold serial dilution, and then inoculated with an appropriate amount of bacterial solution. After incubation for 24 hours, the minimum inhibitory concentration of the compound was observed.

The culture medium used in the experiment was CAMHB broth, CAMHB+5% defibrillated sheep blood broth.

The preserved bacteria strain was inoculated into a serum-supplemented medium and cultured at 37° C. for 16-18 hours. An appropriate amount of bacteria after the completion of subculture and saline were placed into a turbidimetric tube and calibrated to the McFarland standard with a McFarland turbidimeter. The bacterial suspension was diluted 10 times with saline to prepare test bacterial solutions of certain concentrations (5×10⁵ to 5×10⁶ cfu/mL) for later use.

Tylosin and the compounds obtained in the examples were dissolved in methanol to the desired concentration of each compound (1.0 mg/mL), stored in sterilized brown vials, stoppered, and sealed for later use. The working concentration range against Gram-negative bacteria was 0.25 μg/mL to 128 μg/mL; and the working concentration range against Gram-positive bacteria was 0.098 μg/mL to 50 μg/mL.

The 96-well plate micro double dilution method was used. A broth culture medium was added into the 96-well plate, and the prepared compound solution was diluted in a trace two-fold decreasing concentration, so that the concentrations of the compound solutions from the first well to the tenth well showed a two-fold decreasing tendency, and neither the eleventh well nor the twelfth well was added with the compound solution. Finally, the prepared bacterial solution (of a concentration of 5×10⁵ to 5×10⁶ cfu/mL) was added into the first well to the eleventh well, and the twelfth well was not added with the bacterial solution to serve as a blank control. The 96-well plate was placed in a 37° C. incubator, cultured in a static state for 24 hours to observe the bacterial growth in each well. The solution in the well that inhibited bacterial growth was transparent, and the solution in the well that could not inhibit bacterial growth was turbid. The concentration in the well with a transparent solution was selected to be the minimum inhibitory concentration (MIC) of the sample.

The results are shown in the following tables.

TABLE 1
MIC values of the compounds of the present invention (μg/mL)

| | | Streptococcus | Escherichia |
|---|---|---|---|
| | | pneumoniae | coli |
| Example | Compound No. | ATCC 49169 | ATCC 8099 |
| Example 1 | Ia | 0.391 | 128 |
| Example 2 | Ib | 0.781 | 128 |
| Example 3 | Ic | 0.195 | 64 |
| Example 4 | Id | 0.781 | 128 |
| Positive Control | Tylosin | 0.781 | 128 |

The results show that compared with tylosin, the compounds obtained in Examples 1-4 have superior or equivalent in vitro antibacterial activity against Streptococcus pneumoniae (a representative of Gram-positive bacteria) and Escherichia coli (a representative of Gram-negative bacteria), indicating that the compounds represented by Formula I exhibit antibacterial activity against Gram-positive bacteria, a portion of Gram-negative bacteria and mycoplasma.Specifically, the derivative Ia obtained by reaction with 3-amino-1-propanol has slightly better antibacterial effect than tylosin against Streptococcus pneumoniae ATCC 49169, and comparable antibacterial effect to tylosin against Escherichia coli 8099. It is speculated that the group at R-position increases the binding with Streptococcus pneumoniae and improves the antibacterial activity. The derivative Ic obtained by reaction with (R)-prolinol exhibits superior antibacterial effect to tylosin against both Streptococcus pneumoniae ATCC 49169 and Escherichia coli 8099; and the derivative Id, obtained by reaction with (S)-prolinol, introduces an N-containing pyrrole ring, which can further increase the antibacterial activity; however the anticipated increase in activity was not observed, possibly due to steric hindrance.

Although the present invention with general descriptions and specific embodiments has been described in detail, it is obvious to those skilled in the art that some modifications or improvements may be made thereto based on the present invention. Therefore, these modifications or improvements made without departing from the spirit of the present invention fall within the scope claimed by the present invention.

### Industrial Applications

The present invention has the following technical advantages:

The present invention provides a macrolide compound and a preparation method and application thereof. The compound or a pharmaceutically acceptable salt thereof can be used to treat or prevent bacterial or mycoplasma infections, providing more selectivity for animal husbandry and veterinary clinics.

What is claimed is:

1-14. (canceled)

15. A macrolide compound, having a structure represented by Formula Ic:

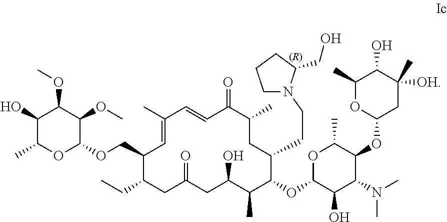

16. A preparation method of the macrolide compound according to claim 15, comprising the following steps:
(A) subjecting tylosin A and an amino alcohol to a reaction in a non-polar solvent to obtain a reaction solution; wherein the amino alcohol is (R)-prolinol; a molar ratio of the amino alcohol to the tylosin A is from 2:1 to 5:1; and the non-polar solvent is toluene; and
(B) adding an acid to the reaction solution, and reacting to obtain a hydroxyl tertiary amino-modified macrolide compound; wherein the acid is formic acid; the acid is added when a temperature of a reaction system reaches 75-85° C.; a molar ratio of the acid to the tylosin A is from 3:1 to 6:1; and
reaction conditions in the step (B) comprise: a temperature of 78-80° C. and a time period of 2-3 h.

17. A veterinary pharmaceutical composition, comprising the macrolide compound according to claim 15.

18. A pharmaceutical preparation, comprising the macrolide compound according to claim 15.

19. A use of the macrolide compound according to claim 15, a veterinary pharmaceutical composition comprising the macrolide compound, or a pharmaceutical preparation comprising the macrolide compound in preparation of an anti-pathogen infection medicament;
wherein a pathogen is Streptococcus pneumoniae and/or Escherichia coli.
